(19) United States
(12) Patent Application Publication
(10) Pub. No.: US 2007/0270848 A1
(43) Pub. Date: Nov. 22, 2007

(54) Interlocking hip screw

(76) Inventor: Shih-Wei Lin, Jhongli City (TW)

Correspondence Address: TROXELL LAW OFFICE PLLC, 5205 LEESBURG PIKE, SUITE 1404, FALLS CHURCH, VA, 22041, US

(21) Appl. No.: 11/414,469

(22) Filed: May 1, 2006

(51) Int. Cl. A61F 2/30

(52) U.S. Cl. 606/65

(57) ABSTRACT

A femur slide nail has one end penetrating into head of the femur through neck from side of the femur and another end fixed to shaft of the femur; a thickening section being disposed to the nail corresponding to the neck; one or multiple transverse holes being provided on the thickening section; and the hole being secured with a fastener so that the nail will not easily breaking away from femur head to improve fixation strength of the nail.

### BACKGROUND OF THE INVENTION

(a) Field of the Invention

The present invention is related to a femur slide nail, and more particularly, to one that prevents the nail will not easily break away from the femur head while improving fixation strength of the nail.

(b) Description of the Prior Art

Whereas the composition of senile people in the structure of population increases by day, more and more cases of osteoporosis are reported. Femur neck fracture and intertrochanteric fracture are two major causes leading to serious complications and even death. Should immediate fixation to facilitate earlier walking and rehabilitation be prescribed for the fracture, complications including bedsore, urinary-tract infection and pneumonia will be soonest invited one after another. A compression hip screw is the most popular treatment for femur fracture. The screw enters from the side of the femur to be implanted through the neck of the femur into the head of the femur, and connected to a side plate to be secured externally to the shaft of the femur.

As illustrated in FIG. 1 of the accompanying drawings, a screw 1 and a side plate 3 mutually hold a fracture of the femur 2 secured in place. To do this, one end of the screw 1 enters from a side of the femur 2 through a neck 21 of the femur 2 into a head 22 of the femur 2; and another end of the screw 1 is fixed to a shaft 23 of the femur 2 by means of the side plate 3. However, in serious case of osteoporosis, the engagement between the screw and the femur head is not necessarily strong enough to support the weight of the patient to result in displacement of the screw in the femur head, and even attract major complications when the screw penetrates through the head of the femur

### SUMMARY OF THE INVENTION

The primary purpose of the present invention is to provide a femur slide nail to secure the nail at where the neck of the femur provides the highest strength to improve fixation strength of the nail without easily displacing in the head of the femur and to minimize failure of operation by giving a thickening section to the diameter of the nail and an additional lateral fastener means to the thickening section.

To achieve the purpose, one end of the nail of the present invention penetrates into the head of the femur through the neck of the femur from one side of the femur while another end is inserted into a side plate and fixed to the shaft of the femur. Wherein, a thickening section is disposed at where appropriately to the nail corresponding to the neck of the femur and one or multiple transverse holes is preset on the thickening section to facilitate the fastener to be secured in the hole for the nail to be fixed at where providing the highest strength in the neck of the femur to improve the fixation results.

### BRIEF DESCRIPTION OF THE DRAWINGS

FIG. 1 is a schematic view showing a construction of having a screw and a side plate of the prior art implanted into a femur.

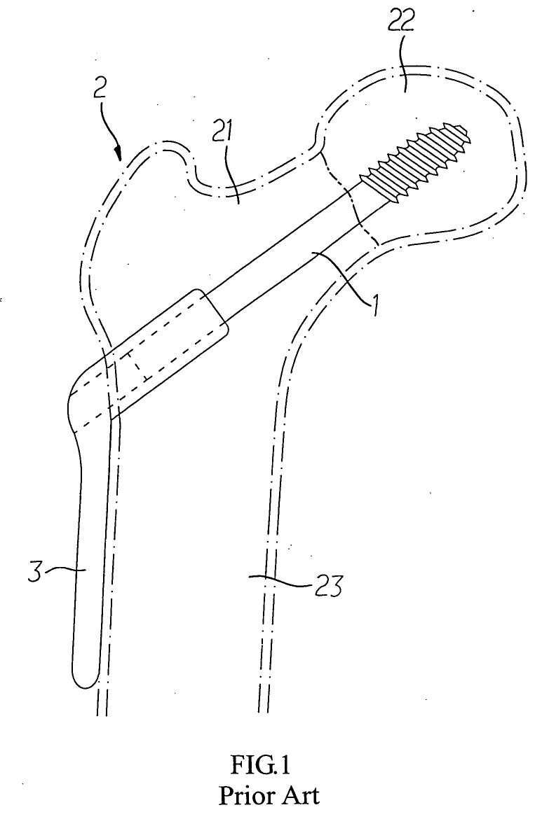

FIG. 2 is an exploded view showing a nail and a side panel of a preferred embodiment of the present invention.

FIG. 3 is a schematic view showing a construction of the nail and the side plate of the preferred embodiment of the present invention.

FIG. 4 is a schematic view showing that the nail and the side plate of the preferred embodiment of the present invention have been implanted into the femur.

FIG. 5 is an exploded view showing a nail and a side panel of another preferred embodiment of the present invention.

FIG. 6 is a schematic view showing that the nail and the side plate of another preferred embodiment of the present invention have been implanted into the femur.

### DETAILED DESCRIPTION OF THE PREFERRED EMBODIMENTS

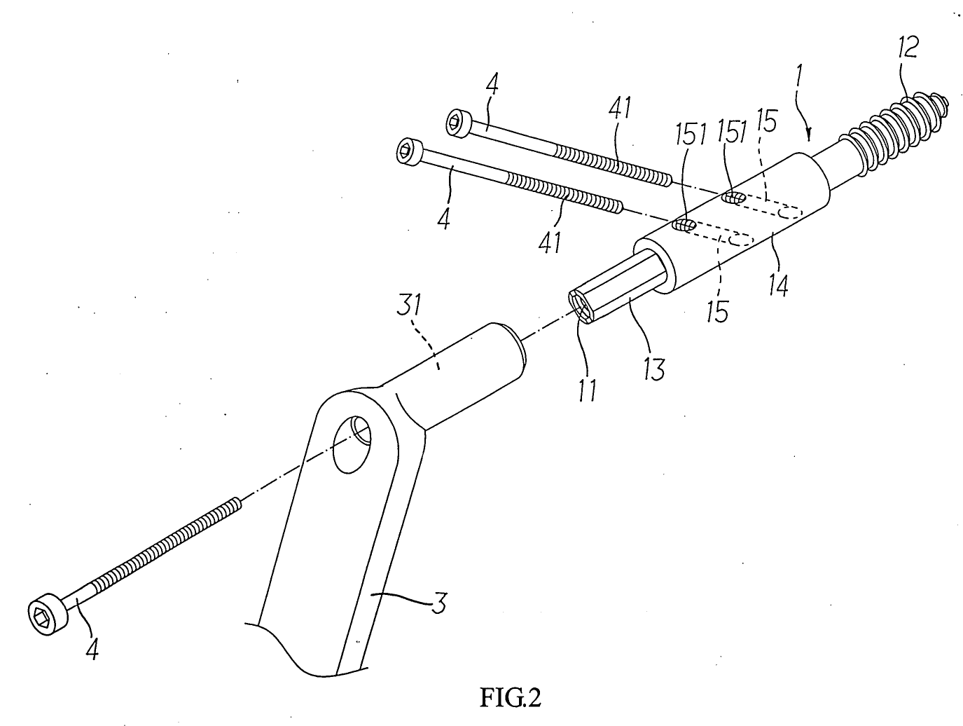

Referring to FIG. 2 for a preferred embodiment of the present invention, a nail 1 made in a cylindrical form contains passage 11. One end of the nail 1 is provided with a threaded section 12 and another end is provided with a connection end 13 in a non-true circular form. A thickening section 14 is made at where between the threaded section 12 and the connection end 13 in ad diameter greater than that of the connection end 13. One or multiple transverse holes 15 is provided on the thickening section 14. Two transverse holes 15 are provided in the preferred embodiment and each is disposed with an internal threaded section 151.

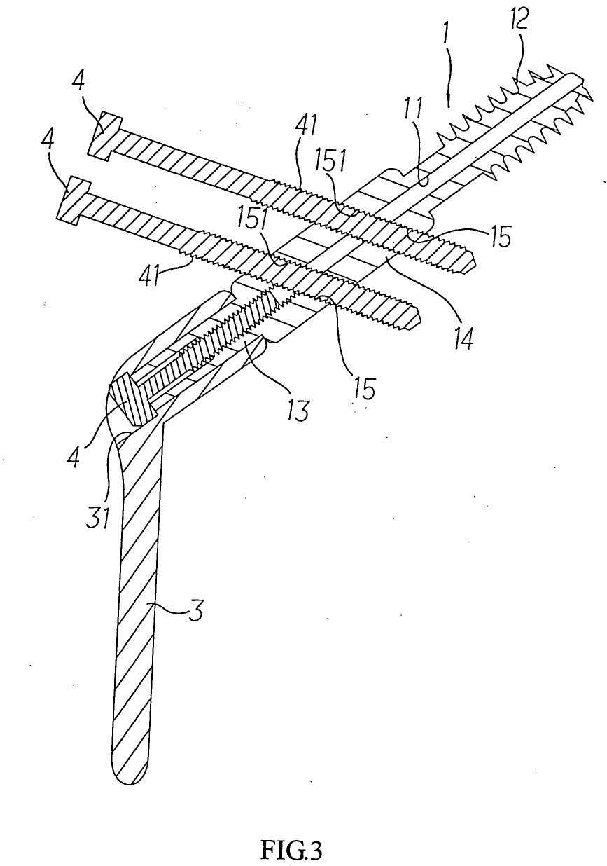

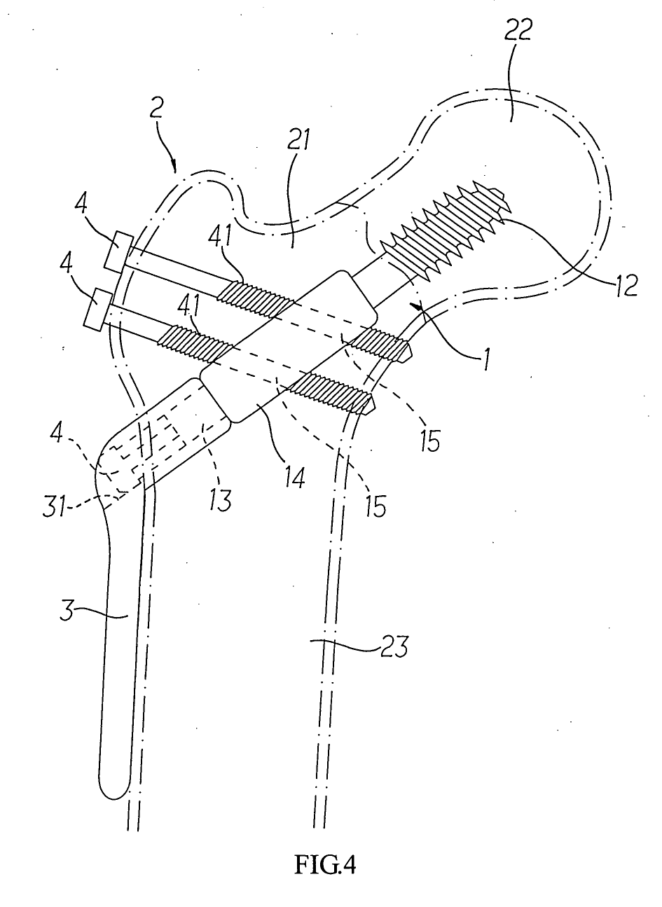

A guide pin (not illustrated) is first inserted from one side of the femur through a neck of the femur into a head of the femur to help alignment of the nail during the implantation procedure of the prior art. The end of the nail 1 with the threaded section 12 is inserted from one side of a femur 2 into a head 22 of the femur 2 through a neck 21 of the femur 2. As also illustrated in FIGS. 3 and 4, a sleeve 31 is provided to the terminal of a side plate 3 to receive insertion by the connection end 13 of the nail 1. The connection end 13 is made in a non-true circular form to prevent from turning inside the sleeve 31. An internal threaded section 131 is provided to the passage 11 at where closer to the connection end 13 to permit insertion of a fastener 4, related to a screw in the preferred embodiment, from the other end of the sleeve to secure the connection end 13 inside the sleeve 31 for both of the nail 1 and the side plate 3 to be firmly secured to each other while the side plate 3 is fixed to a shaft 23 of the femur 2.

To fasteners 4, each related to a screw in the preferred embodiment, are provided for each to be inserted and secured in the transverse hole 15 with an external threaded section 41 of the fastener 4. Whereas the thickening section 14 is located at where the highest strength is provided on the neck 21 of the femur 2 and is secured by means of the fastener 4, the fixation strength of the nail 1 is improved to prevent it from making displacement inside the head 22 of the femur 2 thus to minimize the failure of the operation.

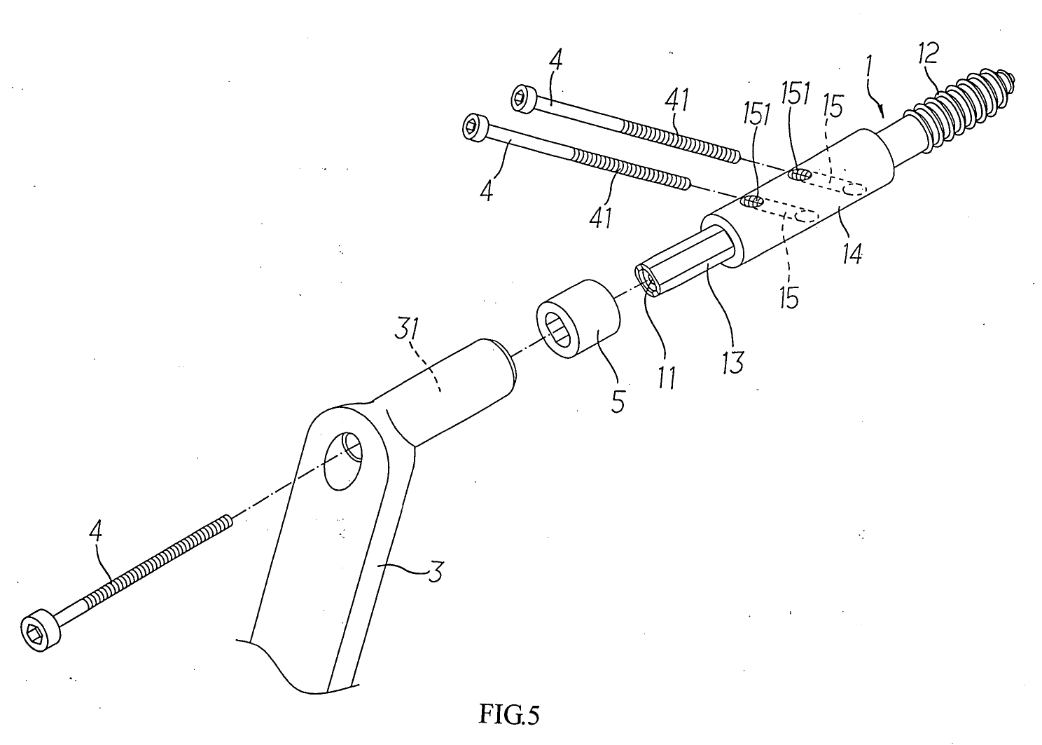

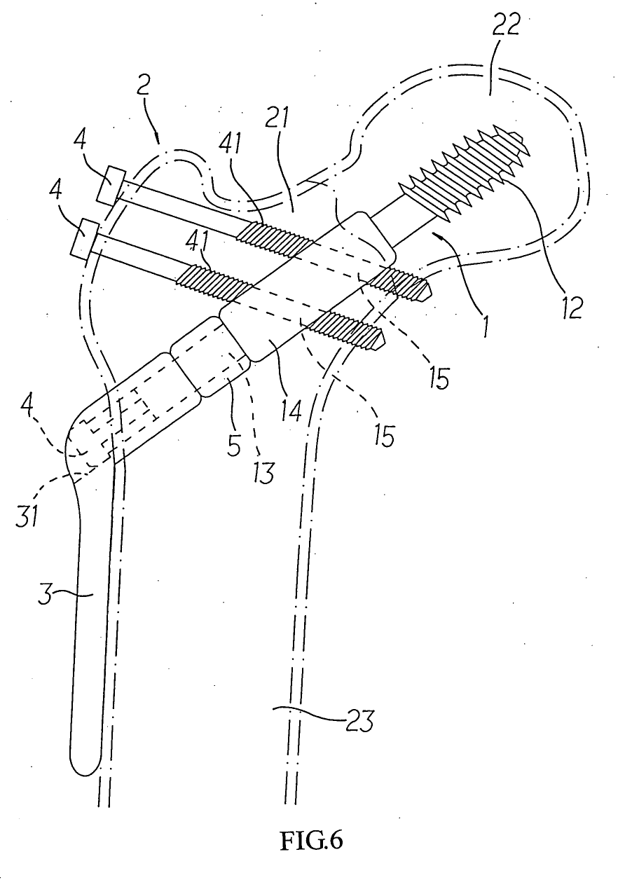

Furthermore, a ring 5 is filled between the connection end 13 and the sleeve 31 as illustrated in FIGS. 5 and 6 as an optional item for the length adjustment. In case of a larger head 22 of the femur 2, the connection end 13 is first inserted into the ring 5 before being secured in the sleeve 31 and finally secured at another end of the sleeve by means of the fastener 4 for both of the nail 1 and the side plate 3 to be secured to each other.

The prevent invention provides an improved structure of a femur slide nail, and the application for a utility patent is duly filed accordingly. However, it is to be noted that the preferred embodiments disclosed in the specification and the accompanying drawings are not limiting the present invention; and that any construction, installation, or characteristics that is same or similar to that of the present invention should fall within the scope of the purposes and claims of the present invention.

I claim:

1. A femur slide nail has one end penetrating into a head of the femur through a neck of the femur from one side of the femur and another end fixed to a shaft of the femur by mean of a side plate; a thickening section being provided to the nail at where appropriately corresponding to the neck of the femur; and one or multiple transverse holes being preset on the thickening section to receive insertion of a fastener to improve fixation strength of the nail.

2. The femur slide nail of claim 1, wherein one end of the nail that penetrates into the head and the neck of the femur is threaded.

3. The femur slide nail of claim 1, wherein the nail relates to a tube containing a passage.

4. The femur slide nail of claim 1, wherein the side plate penetrates into the femur by means of a sleeve disposed at the terminal of the side plate and receives insertion of a connection end of the nail.

5. The femur slide nail of claim 1, wherein the end of the nail penetrating into the head and the neck of the femur is threaded and another end of the nail is formed a connection end to be inserted into the side plate while the thickening section is located at where between the threaded end of the connection end of the nail.

6. The femur slide nail of claim 1, wherein the transverse hole is internally threaded and the fastener is externally threaded for both to mesh to each other.

7. The femur slide nail of claim 1, wherein the fastener relates to a screw.

8. The femur slide nail of claim 4, wherein the connection end is made in a non-true circular form.

9. The femur slide nail of claim 4, wherein a ring is filled between the connection end and the sleeve for length adjustment.

10. The femur slide nail of claim 3, wherein the passage at where close to the connection end of the nail is internally threaded to permit insertion of a fastener to fix from the other end of the sleeve.
